SEQUENCE LISTING (6 sequences)

SEQ ID NO: 1 - protein, 10 aa, Artificial Sequence
  Clusterin peptide
TLLSNLEEAK

SEQ ID NO: 2 - protein, 12 aa, Artificial Sequence
  Clusterin peptide
ASSIIDELFQDR

SEQ ID NO: 3 - protein, 9 aa, Artificial Sequence
  Clusterin peptide
IDSLLENDR

SEQ ID NO: 4 - protein, 23 aa, Artificial Sequence
  Clusterin peptide
VTTVASHTSDSDVPSGVTEVVVK

SEQ ID NO: 5 - protein, 6 aa, Artificial Sequence
  Clusterin peptide
ALQEYR

SEQ ID NO: 6 - protein, 6 aa, Artificial Sequence
  Clusterin peptide
YNELLK

(19) United States
(12) Patent Application Publication
(10) Pub. No.: US 2011/0082187 A1
(43) Pub. Date: Apr. 7, 2011

(54) MARKERS AND METHODS RELATING TO THE ASSESSMENT OF ALZHEIMER'S DISEASE

(76) Inventors: JAMES CAMPBELL, COBHAM (GB); MADHAV THAMBISETTY, SILVER SPRING, MD (US); SIMON LOVESTONE, LONDON (GB)

(21) Appl. No.: 12/878,266

(22) Filed: Sep. 9, 2010

Related U.S. Application Data
(60) Provisional application No. 61/241,507, filed on Sep. 11, 2009.

(51) Int. Cl. A61K 31/7105; G01N 33/50; G01N 33/53; G01N 33/48; A61P 25/28

(52) U.S. Cl. 514/44 A; 436/86; 435/7.1; 436/501; 436/94

(57) ABSTRACT

Use of clusterin as a biomarker of Alzheimer's disease (AD), particularly methods and compositions for detection of clusterin in a biological sample and assessment of in vivo pathology, disease severity and rate of clinical progression in a subject having or suspected of having AD.

### CROSS-REFERENCE TO RELATED APPLICATIONS

The present application claims the benefit of U.S. Provisional Patent Application No. 61/241,507, filed Sep. 11, 2009, the entire disclosure of which is incorporated by reference herein.

### FIELD OF THE INVENTION

The present invention relates to compositions and methods for assessing Alzheimer's disease severity, progression and pathology. In particular, the present invention relates to provision of biologically relevant biomarkers, including biomarkers having prognostic utility.

### BACKGROUND TO THE INVENTION

There is an urgent need for biomarkers of Alzheimer's disease (AD); especially to detect the early stages of disease. Such biomarkers have considerable potential in both clinical practice and research where they may accelerate the development of novel disease-modifying treatments [1]. In both the United States and Europe public/private consortia are conducting studies to discover such biomarkers [2, 3]. The most advanced biomarkers to date in AD are serial structural imaging (MRI) and assays of the candidate proteins, Aβ and tau, in cerebrospinal fluid (CSF) [4]. However, these methodologies are not without their limitations and blood based biomarkers would be of considerable value for use in large, community based or multi-centre studies and in the routine clinical care of large numbers of elderly people.

The most common design in biomarker discovery is to compare samples from disease cases to normal control subjects. This strategy has been used previously in proteomic analysis of plasma to derive a panel of proteins differentiating AD from age-matched control subjects [5]. Using two dimensional gel electrophoresis (2DGE) followed by liquid chromatography tandem mass spectrometry (LC-MS/MS), a panel of plasma proteins were identified whose concentrations were significantly different in AD compared to control subjects. Some of these proteins were then validated by confirming their differential expression in AD using quantitative immunoassays. Subsequently, these proteins were shown to correlate with imaging measures of hippocampal metabolism in patients with AD [6] and have been independently replicated [7, 8]. Others have used large protein arrays to successfully identify panels of plasma proteins associated with disease using this experimental design [9].

Although this standard approach, relying upon the binary distinction of differentiating disease from control has proven productive, it may not be suited to the identification of biomarkers aiming to detect early disease states, especially in a disease such as AD with a long pre-clinical prodrome. Many controls in these studies will therefore have AD pathology even in the absence of clinical symptoms. However, preclinical detection and treatments will require biomarkers reflecting disease state, independent of clinical symptoms, and ideally before the onset of any symptoms [10]. Furthermore, current approaches to biomarker discovery in AD do not address the considerable heterogeneity in disease progression in patients with established AD [11, 12]. The predominant biomarker discovery study design, comparing cases to controls, is therefore unlikely to identify biomarkers reflecting early disease pathology or disease progression. Such markers are essential for use in clinical practice and would be invaluable in clinical trials for the enrichment of at-risk patient populations and for patient stratification.

### SUMMARY OF THE INVENTION

The present inventors have employed a different approach to the discovery of blood-based AD biomarkers in which plasma proteins that reflect the extent of in vivo disease pathology and rate of clinical progression were sought rather than binary distinctions between case and control. As described in detail herein, the present inventors have found that clusterin (also known as Apolipoprotein J or apoJ) is a biologically relevant peripheral biomarker of AD, with higher levels being associated with more aggressive AD. In particular, elevated blood plasma concentration of clusterin is associated with brain atrophy, cognitive impairment and speed of progression of AD.

Accordingly, in a first aspect the present invention provides a method of assessing, including prognosing, Alzheimer's disease (AD) in a subject, the method comprising:
determining an amount (e.g. concentration) of clusterin in a biological sample obtained from said subject, wherein a greater amount of clusterin is indicative of said subject having an aggressive form of AD and/or a poor prognosis. In some cases of the method of this aspect of the invention, a greater amount of clusterin is indicative of said subject having rapidly progressing AD, more severe cognitive impairment and/or more severe brain pathology.

The method according this and other aspects of the invention may comprise comparing said amount of clusterin with a reference level. In light of the present disclosure, the skilled person is readily able to determine a suitable reference level, e.g. by deriving a mean and range of values from samples derived from a population of subjects. In some cases, the method of this and other aspects of the invention may further comprise determining a reference level above which the amount of clusterin can be considered to indicate an aggressive form of AD and/or a poor prognosis, particularly rapidly progressing AD, more severe cognitive impairment and/or more severe brain pathology. However, the reference level is preferably a pre-determined value, which may for example be provided in the form of an accessible data record. The reference level may be chosen as a level that discriminates more aggressive AD from less aggressive AD, particularly a level that discriminates rapidly progressing AD (e.g. a decline in a mini-mental state examination (MMSE) score of said subject at a rate of at least 2 MMSE points per year; and/or a decline in an AD assessment scale-cognitive (ADAS-Cog) score of said subject at a rate of at least 2 ADAS-Cog points per year) from non-rapidly progressing AD (e.g. a decline in an MMSE score of said subject at a rate of not more than 2 MMSE points per year; and/or a decline in an ADAS-Cog score of said subject at a rate of not more than 2 ADAS-Cog points per year). Preferably, the reference level is a value expressed as a concentration of clusterin in units of mass per unit volume of a liquid sample or unit mass of a tissue sample. For example, the reference level may be expressed as μg of clusterin per ml of a bodily fluid.

In accordance with the method of this and other aspects of the invention, the biological sample may comprise blood plasma, blood cells, serum, saliva, cerebro-spinal fluid (CSF) or a tissue biopsy. Preferably, the biological sample has previously been isolated or obtained from the subject. The biological sample may have been stored and/or processed (e.g. to remove cellular debris or contaminants) prior to determining the amount (e.g. concentration) of clusterin in the sample. However, in some cases the method may further comprise a step of obtaining the biological sample from the subject and optionally storing and/or processing the sample prior to determining the amount (e.g. concentration) of clusterin in the sample. Preferably, the biological sample comprises blood plasma and the method comprises quantifying the blood plasma concentration of clusterin (e.g. in terms of μg clusterin per ml of blood plasma). When the biological sample comprises blood plasma and determining the amount of clusterin comprises quantifying the blood plasma concentration of clusterin, the reference level may be chosen as a clusterin concentration that discriminates more aggressive AD from less aggressive AD. For example, with reference to FIGS. 3A and 3B, the reference level for human subjects when clusterin amount is determined as a concentration of clusterin per unit volume of blood plasma sample may be in the range of about 80 μg/ml to about 85 μg/ml (e.g., 80, 81, 82, 83, 84 or 85 μg/ml). In some cases the reference level may be chosen according to the assay used to determine the amount of clusterin. A reference level in this range may represent a threshold dividing subjects into those below who are more likely to have a less aggressive form of AD (e.g. non-rapidly progressing AD) from those above who are more likely to have a more aggressive form of AD (e.g. rapidly progressing AD). However, the reference level may be a value that is typical of a less aggressive form of AD (e.g. non-rapidly progressing AD), in which case a subject having a reading significantly above the reference level may be considered as having or probably having an aggressive form of AD (e.g. rapidly progressing AD). Whereas the reference level may be a value that is typical of a more aggressive form of AD (e.g. rapidly progressing AD), in which case a subject having a reading significantly below the reference level may be considered as having or probably having a less aggressive form of AD (e.g. non-rapidly progressing AD).

In accordance with the method of this and other aspects of the invention, the method may further comprise determining one or more additional indicators of risk of AD, severity of AD, course of AD (such as rate or extent of AD progression). Such additional indicators may include one or more (such as 2, 3, 4, 5 or more) indicators selected from: brain imaging results (including serial structural MRI), cognitive assessment tests (including MMSE or ADAS-Cog), APOE4 status (particularly presence of one or more APOE4 ε4 alleles), fibrillar amyloid burden (particularly fibrillar amyloid load in the entorhinal cortex and/or hippocampus), CSF levels of Aβ and/or tau, presence of mutation in an APP gene, presence of mutation in a presenilin gene and presence of mutation in a clusterin gene. In some cases the method in accordance with this and other aspects of the invention is used as part of a panel of assessments for diagnosis, prognosis and/or treatment monitoring in a subject having or suspected of having AD.

In accordance with the method of this and other aspects of the invention, determining the amount of clusterin in the biological sample may be achieved using any suitable method.

The determination may involve direct quantification of clusterin mass or concentration. The determination may involve indirect quantification, e.g. using an assay that provides a measure that is correlated with the amount (e.g. concentration) of clusterin. In certain cases of the method of this and other aspects of the invention, determining the amount of clusterin comprises:
contacting said sample with at least one specific binding member that selectively binds to clusterin; and
detecting and/or quantifying a complex formed by said specific binding member and clusterin.

The specific binding member may be an antibody or antibody fragment that selectively binds clusterin. For example, a convenient assay format for determination of clusterin concentration is an ELISA, such as the human clusterin ELISA kit, RD194034200R, available from Biovendor Laboratory Medicine Inc, Modrice, Czech Republic. The determination may comprise preparing a standard curve using standards of known clusterin concentration and comparing the reading obtained with the sample from the subject with the standard curve thereby to derive a measure of the clusterin concentration in the sample from the subject. A variety of methods may suitably be employed for determination of clusterin amount (e.g. concentration), non-limiting examples of which are: Western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), liquid immunoarray technology (e.g. Luminex xMAP technology or Becton-Dickinson FACS technology), immunocytochemical or immunohistochemical techniques, techniques based on the use protein microarrays that include specific antibodies, “dipstick” assays, affinity chromatography techniques and ligand binding assays. The specific binding member may be an antibody or antibody fragment that selectively binds clusterin. Any suitable antibody format may be employed, as described further herein. A further class of specific binding members contemplated herein in accordance with any aspect of the present invention comprises aptamers (including nucleic acid aptamers and peptide aptamers). Advantageously, an aptamer directed to clusterin may be provided using a technique such as that known as SELEX (Systematic Evolution of Ligands by Exponential Enrichment), described in U.S. Pat. Nos. 5,475,096 and 5,270,163.

In some cases of the method in accordance with this and other aspects of the invention, the determination of the amount of clusterin comprises measuring the level of clusterin-derived peptide by mass spectrometry. Techniques suitable for measuring the level of a clusterin-derived peptides by mass spectrometry are readily available to the skilled person and include techniques related to Selected Reaction Monitoring (SRM) and Multiple Reaction Monitoring (MRM)isotope dilution mass spectrometry including SILAC, AQUA (as disclosed in WO 03/016861; the entire contents of which is specifically incorporated herein by reference) and TMTcalibrator (as disclosed in WO 2008/110581; the entire contents of which is specifically incorporated herein by reference). WO 2008/110581 discloses a method using isobaric mass tags to label separate aliquots of all proteins in a reference plasma sample which can, after labelling, be mixed in quantitative ratios to deliver a standard calibration curve. A patient sample is then labelled with a further independent member of the same set of isobaric mass tags and mixed with the calibration curve. This mixture is then subjected to tandem mass spectrometry and peptides derived from specific proteins can be identified and quantified based on the appearance of unique mass reporter ions released from the isobaric mass tags in the MS/MS spectrum. FIG. 9 of WO 2008/110581 shows a calibration curve for the measurement of clusterin in normal plasma. The clusterin-derived peptide may comprise an amino acid sequence of at least 5, 6, 7, 10, 15 or 20 contiguous amino acids that has at least 80%, 90%, 95%, 99% or 100% identity to a known or predicted fragment of the human clusterin amino acid sequence disclosed in UniProt Accession No. P10909, sequence version 1 and GI No. 116533 or such appropriate homologue of clusterin in non-human subjects. The known or predicted fragment may be a fragment created by trypsin or Lys-C digestion of said clusterin protein. In some cases, when employing mass spectrometry-based determination of clusterin, the method of this and other aspects of the invention comprises providing a calibration sample comprising at least two different aliquots comprising clusterin and/or at least one clusterin-derived peptide, each aliquot being of known quantity and wherein said biological sample and each of said aliquots are differentially labelled with one or more isobaric mass labels. Preferably, the isobaric mass labels each comprise a different mass spectrometrically distinct mass marker group. In particular, the mass labels may be as defined in WO 2008/110581 (e.g. having the structure X-L-M as defined therein).

In some cases of the method in accordance with this and other aspects of the invention, the determination of the amount of clusterin comprises measuring the level of a clusterin-encoding mRNA derived from the biological sample. As described further herein, the level of clusterin-encoding mRNA in blood cells isolated from subjects having AD has been found to be elevated compared with non-AD controls and with subjects having MCI, but not AD. Furthermore, there is a well-recognised link between increased gene expression in a source tissue and increased amount, including increased concentration, of the protein encoded by the gene in one or more bodily tissues or bodily fluids. Therefore, an indirect determination of clusterin amount may be provided by measuring the level of clusterin-encoding mRNA in a biological sample (e.g. a sample comprising cells isolated from the brain, cells isolated from the liver or blood cells). Techniques suitable for measuring the level of a clusterin-encoding mRNA are readily available to the skilled person and include quantitative or “real time” reverse transcriptase PCR or Northern blots. The method of measuring the level of a clusterin-encoding mRNA may comprising using at least one primer or probe that is directed to the sequence of a clusterin-encoding gene, or the compelement thereof. The at least one primer or probe may comprise a nucleotide sequence of at least 10, 15, 20, 25, 30 or 50 contiguous nucleotides that has at least 70%, 80%, 90%, 95%, 99% or 100% identity to a nucleotide sequence encoding the human clusterin amino acid sequence disclosed in UniProt Accession No. P10909, sequence version 1 and GI No. 116533, calculated over the length of said primer or probe. Preferably, said at least one primer or probe hybridises under stringent conditions to a clusterin-encoding gene, or the complement thereof. Preferably, said clusterin-encoding gene comprises a nucleotide sequence having at least 70%, 80%, 90%, 95%, 99% or 100% identity to a nucleotide sequence of the human clusterin gene disclosed in NCBI GeneID: 1191; or a homologue thereof from a non-human animal (e.g. the murine clusterin gene having NCBI GeneID: 12759).

In accordance with the method of this and other aspects of the invention the subject may have been previously diagnosed with AD and/or previously diagnosed with mild cognitive impairment (MCI). The subject is preferably a human. The subject may be a human of at least 60 years of age, optionally at least 70 or at least 80 years of age.

The method in accordance with this and other aspects of the invention may be for assessing (e.g. prognosing) AD in a plurality of subjects, wherein the method comprises determining an amount of clusterin in a biological sample obtained from each of said plurality of subjects. In this way the plurality of subjects may be stratified according to aggressiveness of AD based on the determination of said clusterin amount. In some cases the plurality of subjects are divided into slow progressing AD category and fast progressing AD category based on the determination of said clusterin amount.

In accordance with the method of this and other aspects of the invention the determination of clusterin amount may be used to inform the treatment strategy of said subject or subjects and/or to monitor the success of a treatment strategy. Thus, a relatively high amount of clusterin (e.g. above a reference level as described above) may indicate an aggressive form of AD (such as rapidly progressing AD) and therefore a poor prognosis which finding may be used to recommend, or to proceed with, an aggressive therapeutic strategy. Subjects placed in a rapidly progressing category of AD, in particular, may warrant follow-up assessment, e.g. to monitor the success or failure of a therapeutic strategy.

The method in accordance with this and other aspects of the invention is fully applicable to subjects whether or not they have an aggressive form of AD. However, in some cases it is envisaged that the determination of clusterin amount does indicate that said subject or subjects have rapidly progressing AD, more severe cognitive impairment and/or more severe brain pathology.

In accordance with this and other aspects of the invention, cognitive impairment (including “rapidly progressing AD” or “non-rapidly progressing AD”) may be determined by an established measure of AD-related impairment. For example, there are four commonly accepted domains of dementia-COGNITION (typically measured with MMSE and ADAS-cog as discussed further herein), BEHAVIOUR (typically measured with Neuropsychiatric inventory (NPI)), FUNCTION (typically measured with scales such as Disability Assessment for Dementia (DAD) or Bristol Activities of daily living (bADL)) and GLOBAL SEVERITY typically measured with the functional assessment scale in dementia (FAST) or Clinical Dementia Rating scale (CDR). These scales have been described previously [36-38; the entire contents of which are expressly incorporated herein by reference for all purposes].

In some cases in accordance with this and other aspects of the invention, “rapidly progressing AD” may be AD characterised by rapid decline: in a mini-mental state examination (MMSE) score of the subject at a rate of at least 2 MMSE points per year; and/or a decline in an AD assessment scale-cognitive (ADAS-Cog) score of the subject at a rate of at least 2 ADAS-Cog points per year. Both retrospective decline (i.e. the rate of decline prior to the point at which the sample is obtained from the subject) and prospective decline (i.e. the rate of decline following the point at which the sample is obtained from the subject) are specifically contemplated. In other words, the method of this and other aspects of the invention may indicate that the subject has rapidly progressing AD, which indication may be of a prospective and/or retrospective rate of decline of at least 2 MMSE points and/or 2 ADAS-Cog points per year. Where a scale other than MMSE or ADAS-Cog is employed (e.g. a scale as described above), the rate of decline may be a rate of decline that corresponds to or substantially translates to a rate of decline of 2 MMSE points per year and/or 2 ADAS0Cog points per year.

In accordance with this and other aspects of the invention “more severe brain pathology” may be a more severe brain pathology selected from: fibrillar amyloid burden in the entorhinal cortex (ERC), atrophy of the ERC and atrophy of the hippocampus. In some cases, the method of this and other aspects of the invention a determination of a greater amount of clusterin in said sample (e.g. an amount above a reference level as defined above) may indicate that the subject has a decrease in hippocampal volume of at least 10%, 20%, 30%, 40%, 50% or more, as assessed by MRI. In some cases, the method of this and other aspects of the invention a determination of a greater amount of clusterin in said sample (e.g. an amount above a reference level as defined above) may indicate that the subject has a decrease in ERC volume of at least 10%, 20%, 30%, 40%, 50% or more, as assessed by MRI.

In accordance with this and other aspects of the invention “clusterin” refers to a full-length clusterin protein or an isoform, fragment, truncated form, orthologue, paralogue, derivative or variant thereof. Preferably, the clusterin comprises or consists of an amino acid sequence having at least 70%, 80%, 90%, 95%, 99% or 100% identity to the human clusterin sequence disclosed in UniProt Accession No. P10909, sequence version 1 and GI No. 116533, calculated over the full length of said human clusterin sequence; or a fragment thereof comprising at least 50, 100, 150, 200, 250, 300, 350, 400, 425 or 449 contiguous amino acids. In some cases, the clusterin is detectable by the anti-clusterin antibody of the RD194034200R human clusterin ELISA kit obtainable from Biovendor Laboratory Medicine Inc, Modrice, Czech Republic. However, as described further herein the subject may be non-human (e.g. a laboratory animal for use in screening). Therefore, the clusterin may be the clusterin protein of the same species as the subject. Accordingly, the clusterin may be rodent, particularly murine, clusterin, e.g. that comprises or consists of an amino acid sequence having at least 70%, 80%, 90%, 95%, 99% or 100% identity to the mouse clusterin sequence disclosed in UniProt Accession No. Q06890, sequence version 1, GI: 729152, calculated over the full length of said mouse clusterin sequence; or a fragment thereof comprising at least 50, 100, 150, 200, 250, 300, 350, 400, 425 or 448 contiguous amino acids.

As described herein, elevated clusterin is associated with more aggressive AD (e.g. rapidly progressing AD) and clusterin exhibits altered gene expression in AD. Taken together with previous work reporting a role for clusterin in regulating in vivo amyloidogenesis, the present inventors contemplate, without wishing to be bound by theory, that raised clusterin may be an aetiopathological event, rather than simply a reaction to other pathology in AD. Therefore, clusterin may represent an important biomarker for developing and/or monitoring putative AD therapies.

Accordingly, in a second aspect the present invention provides a method for screening a test agent to determine its usefulness in treating AD, the method comprising:
determining an amount of clusterin in a biological sample obtained from a test subject having at least one AD-related clinical or pathological feature, which test subject has been treated with the test agent; and
comparing the determination of said clusterin amount with a control amount, which corresponds to the amount of clusterin in a biological sample obtained from a control subject having at least one Alzheimer's disease-related clinical or pathological feature, which control subject has not been treated with the test agent,
whereby the test agent is selected or rejected according to the extent to which the test agent alters said clusterin amount relative to said control amount. Preferably, the test agent is found to decrease the amount of clusterin relative to said control amount.

In accordance with this aspect of the invention, determining an amount of clusterin may be as defined above in relation to the first aspect of the invention. Moreover, the subject, the clusterin, the biological sample and/or the reference level may be as defined above in relation to the first aspect of the invention. Preferably, the biological sample comprises blood plasma and determining the amount of clusterin comprises quantifying the blood plasma concentration of clusterin.

In some cases of the method of this aspect of the invention the test subject and the control subject are the same subject, and said control amount corresponds to the amount of clusterin in a biological sample obtained from said subject prior to said subject being treated with the test agent. In this way the method may be used to assess the ability of the test agent to alter or restore clusterin levels in the subject, preferably to a level that is not associated with aggressive AD. In some cases the test subject and the control subject are different. However, the test subject and the control subject may be of the same species, may be age-matched, may have a similar baseline level of clusterin in a biological sample, may have a similar degree or severity of AD clinical state and/or may have a similar degree or severity of AD pathology.

In some cases the test subject may be a subject that or who has been assessed by the method in accordance with the first aspect of the invention. For example, the test subject may have been determined to have an amount of clusterin in the biological sample that indicates the subject has rapidly progressing AD, more severe cognitive impairment and/or more severe brain pathology. Such a subject may be expected to have a more pronounced clinical and/or pathological state and therefore provide a more robust model for testing potential therapeutic agents.

In some cases of the method in accordance with this aspect of the test subject and the control subject are human subjects with AD. In this way the method may be used in the context of human clinical trials, e.g. providing a relatively non-invasive (e.g. simple blood test) way of monitoring the effect of the test agent on AD pathology and/or clinical state.

In some cases of the method in accordance with this aspect of the test subject and the control subject are selected from: mutant amyloid precursor protein (APP) transgenic mice; presenilin-1 (PS-1) transgenic mice; and double transgenic APP/PS-1 transgenic mice. For example, the test subject and the control subject may be TASTPM transgenic mice that overexpress hAPP695swe and presenilin-1 M146V. Such transgenic mice mimic a number of clinical and pathological features of AD, and are useful in pre-clinical studies of putative therapies for AD. The method of this aspect of the invention may provide a relatively simple way of monitoring the effect of the test agent on AD pathology and/or clinical state. Conventional approaches to assessing the effectiveness of candidate therapies for AD may involve invasive or even life-ending protocols (e.g. fixation and staining of rodent brain to assess AD-related pathology). It is envisaged that use of clusterin as a biomarker of AD pathology and/or clinical state may allow a reduction in experimental animal usage in in vivo screening. In particular, when the biological sample is a blood sample, the assessment of AD pathology and/or clinical state by analysis of clusterin levels may permit repeated measurements from the same experimental animal (e.g. serial blood sampling from each transgenic mouse), thereby reducing costs and the number of experimental animals necessary. This has clear advantages in the context of academic and industrial research for AD-targeted therapies.

In a third aspect the present invention provides a method of making a pharmaceutical composition, comprising having identified a test agent using a method in accordance with the second aspect, the further step of manufacturing the test agent and formulating it with a pharmaceutically acceptable carrier to provide the pharmaceutical composition. The test agent or the pharmaceutical composition may be used in the manufacture of a medicament for the treatment of AD. In some cases the test agent is an antagonist or inhibitor of clusterin, such as an anti-clusterin antibody.

In a fourth aspect the present invention provides a kit for assessing AD in a subject in accordance with the method of any aspect of the invention, the kit comprising:
(i) a specific binding member that selectively binds clusterin; and/or at least one primer or probe directed to a nucleic acid sequence that encodes clusterin or which is complementary thereto; and/or at least one standard curve comprising two or more concentrations of a clusterin-derived peptide labelled with a set of isobaric mass tags and an additional member of the same isobaric mass tag set for labelling of a subject-derived sample;
(ii) instructions for performing a method according to the method of any aspect of the invention; and
optionally, (iii) one or more reagents or controls for use in determining an amount of clusterin in a biological sample.

The specific binding member may be an antibody or antibody fragment that selectively binds clusterin, as disclosed herein. The at least one primer or probe may comprise a nucleotide sequence of at least 10, 15, 20, 25, 30 or 50 contiguous nucleotides that has at least 70%, 80%, 90%, 95%, 99% or 100% identity to a nucleotide sequence encoding the human clusterin amino acid sequence disclosed in UniProt Accession No. P10909, sequence version 1 and GI No. 116533, calculated over the length of said primer or probe. The clusterin-derived peptide may comprise an amino acid sequence of at least 5, 6, 7, 10, 15 or 20 contiguous amino acids that has at least 70%, 80%, 90%, 95%, 99% or 100% identity to a known or predicted fragment of the human clusterin amino acid sequence disclosed in UniProt Accession No. P10909, sequence version 1 and GI No. 116533 or such appropriate homologue of clusterin in non-human subjects, and wherein the known or predicted fragment is one created by trypsin or Lys-C digestion of said clusterin protein.

The present invention includes the combination of the aspects and preferred features described except where such a combination is clearly impermissible or is stated to be expressly avoided. These and further aspects and embodiments of the invention are described in further detail below and with reference to the accompanying examples and figures.

### DESCRIPTION OF THE FIGURES

FIG. 1 shows a schematic diagram of the design of A) Discovery and B) Validation-phase studies for the identification of blood-based AD biomarkers associated with both in vivo disease pathology as well as rate of disease progression. C) Association of plasma clusterin concentration with brain amyloid burden was tested in both older humans and a transgenic mouse model of AD;

FIG. 2 shows proteomic identification of plasma proteins associated with hippocampal volume in AD+MCI subjects (top panel) and those associated with fast AD progressors (bottom panel). A representative 2DGE gel is shown with spots outlined denoting proteins associated with hippocampal volume in AD+MCI and proteins associated with fast AD progression;

FIG. 3 shows that increased concentration of plasma clusterin is associated with rate of clinical progression in AD. AD patients with a rapid progression rate, measured A) Prior to blood sampling, and B) One year after blood sampling have significantly increased clusterin concentration relative to slow progressors. C) High levels of clusterin are associated with a significantly greater risk of accelerated cognitive decline subsequent to blood sampling. AD patients (N=204) were assigned a prognostic index derived as their plasma clusterin concentration multiplied by its corresponding regression coefficient (S) in a Cox proportional regression analysis. The figure shows the cumulative hazard functions for the effect of the ‘prognostic factor’ (i.e. plasma clusterin concentration) on the ‘survival probability’ i.e. maintaining a non-aggressive clinical course (decline in MMSE 2 points/year). The cumulative survival functions represent estimated survival probabilities for three representative AD patients with the lowest (5.87 mcg/ml), median (76.84 mcg/ml) and highest plasma clusterin (159 mcg/ml) concentrations showing that an AD patient with the highest clusterin concentration has the lowest probability of maintaining a non-aggressive clinical course one year after sampling. The reported hazard ratio for a 10 mcg/ml rise in plasma clusterin concentration for risk of becoming a rapid AD decliner was 1.071, 95% CI (1-1.147), p=0.05;

FIG. 4 shows A) Gene expression of clusterin is altered in AD. Clusterin mRNA levels are significantly elevated in blood cells from AD patients relative to healthy controls (** p<0.001) and MCI subjects (**p=0.008) after correcting for age. B) Transgenic TASTPM mice overexpressing both human APP and PS1 genes have higher plasma concentration of clusterin relative to wild type littermates at 6 months of age. Inset shows hippocampal and cortical amyloid plaques in a 6-month old TASTPM mouse stained by a monoclonal antibody against Aβ1-42. Wild type mice show no amyloid pathology at this age (not shown).

### DETAILED DESCRIPTION OF THE INVENTION

### Clusterin

Clusterin (Apolipoprotein J; SP-40, 40; TRPM-2; SGP-2; pADHC-9; CLJ; T64; GP III; XIP8) is a highly conserved disulfide-linked secreted heterodimeric glycoprotein of 75-80 kDa but truncated forms targeted to nucleus have also been identified. The protein is constitutively secreted by a number of cell types including epithelial and neuronal cells and is a major protein in physiological fluids including plasma, milk, urine, cerebrospinal fluid and semen. Two recent genome-wide association studies have identified risk loci for AD within, inter alia, CLU, the gene encoding clusterin [34, 35].

However, previous reports on clusterin as a candidate AD biomarker have been inconclusive [23, 25].

As used herein “clusterin” refers to a full-length clusterin protein or an isoform, fragment, truncated form, orthologue, paralogue, derivative or variant thereof. Preferably, the clusterin comprises or consists of an amino acid sequence having at least 70%, 80%, 90%, 95%, 99% or 100% identity to the human clusterin sequence disclosed in UniProt Accession No. P10909, sequence version 1 and GI No. 116533 (incorporated herein by reference in its entirety), calculated over the full length of said human clusterin sequence; or a fragment thereof comprising at least 50, 100, 150, 200, 250, 300, 350, 400, 425 or 449 contiguous amino acids. In some cases, the clusterin is detectable by the anti-clusterin antibody of the RD194034200R human clusterin ELISA kit obtainable from Biovendor Laboratory Medicine Inc, Modrice, Czech Republic. In certain cases the clusterin may be rodent, particularly murine, clusterin, e.g. clusterin protein that comprises or consists of an amino acid sequence having at least 70%, 80%, 90%, 95%, 99% or 100% identity to the mouse clusterin sequence disclosed in UniProt Accession No. Q06890, sequence version 1, GI: 729152 (incorporated herein by reference in its entirety), calculated over the full length of said mouse clusterin sequence; or a fragment thereof comprising at least 50, 100, 150, 200, 250, 300, 350, 400, 425 or 448 contiguous amino acids. The clusterin gene may be the human clusterin gene disclosed in NCBI GeneID: 1191; or a homologue thereof from a non-human animal (e.g. the murine clusterin gene having NCBI GeneID: 12759).

### Biological Sample

As used herein “biological sample” refers to any biological liquid, cellular or tissue sample isolated or obtained from the subject. The biological sample may comprise blood plasma, blood cells, serum, saliva, cerebro-spinal fluid (CSF) or a tissue biopsy. The biological sample may have been stored (e.g. frozen) and/or processed (e.g. to remove cellular debris or contaminants) prior to determining the amount (e.g. concentration) of clusterin in the sample.

### Assaying Clusterin

The determination of an amount of clusterin in the biological sample may involve direct quantification of clusterin mass or concentration or indirect quantification, e.g. using an assay that provides a measure that is correlated with the amount (e.g. concentration) of clusterin. The methods and kits of the present invention may employ immunological detection of clusterin as defined herein. A wide range of immunological assays are available to detect and quantify formation of specific antigen-antibody complexes; numerous competitive or non-competitive protein-binding assays have been described previously and a large number of these are commercially available. A preferred technique involves using a specific binding member that binds clusterin and which may be detected or labelled in order to quantify the amount (e.g. concentration) of clusterin in the sample. For example, an ELISA, such as the human clusterin ELISA kit, RD194034200R, available from Biovendor Laboratory Medicine Inc, Modrice, Czech Republic. However, a variety assay formats are suitable for determination of clusterin amount (e.g. concentration). These include: Western blot, ELISA (Enzyme-Linked Immunosorbent assay), RIA (Radioimmunoassay), Competitive EIA (Competitive Enzyme Immunoassay), DAS-ELISA (Double Antibody Sandwich-ELISA), liquid immunoarray technology (e.g. Luminex xMAP technology or Becton-Dickinson FACS technology), immunocytochemical or immunohistochemical techniques, techniques based on the use protein microarrays that include specific antibodies, “dipstick” assays, affinity chromatography techniques and ligand binding assays. The specific binding member as used herein is preferably an antibody or antibody fragment as defined further herein.

The determination of the amount of clusterin may involve measuring the level of a clusterin-encoding mRNA derived from the biological sample. Techniques suitable for measuring the level of a clusterin-encoding mRNA include quantitative or “real time” reverse transcriptase PCR or Northern blots.

The determination of the amount of clusterin may involve measuring the level of clusterin-derived peptide by mass spectrometry. Techniques suitable for measuring the level of a clusterin-derived peptides by mass spectrometry are readily available to the skilled person and include techniques related to Selected Reaction Monitoring (SRM) and Multiple Reaction Monitoring (MRM)isotope dilution mass spectrometry. The clusterin-derived peptide as used herein is preferably a tryptic digest peptide as defined further herein.

### Antibodies

As used herein with reference to all aspects of the invention, the term “antibody” or “antibody molecule” includes any immunoglobulin whether natural or partly or wholly synthetically produced. The term “antibody” or “antibody molecule” includes monoclonal antibodies and polyclonal antibodies (including polyclonal antisera). Antibodies may be intact or fragments derived from full antibodies (see below).

Antibodies may be human antibodies, humanised antibodies or antibodies of non-human origin. “Monoclonal antibodies” are homogeneous, highly specific antibody populations directed against a single antigenic site or “determinant” of the target molecule. “Polyclonal antibodies” include heterogeneous antibody populations that are directed against different antigenic determinants of the target molecule. The term “antiserum” or “antisera” refers to blood serum containing antibodies obtained from immunized animals.

It has been shown that fragments of a whole antibody can perform the function of binding antigens. Thus reference to antibody herein, and with reference to the methods, arrays and kits of the invention, covers a full antibody and also covers any polypeptide or protein comprising an antibody binding fragment. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site; (viii) bispecific single chain Fv dimers (WO 93/11161) and (ix) “diabodies”, multivalent or multispecific fragments constructed by gene fusion (WO94/13804; 58). Fv, scFv or diabody molecules may be stabilised by the incorporation of disulphide bridges linking the VH and VL domains. Minibodies comprising a scFv joined to a CH3 domain may also be made.

In relation to a “specific binding member”, such as an antibody molecule, the term “selectively binds” may be used herein to refer to the situation in which one member of a specific binding pair will not show any significant binding to molecules other than its specific binding partner(s). The term is also applicable where e.g. an antigen-binding site is specific for a particular epitope that is carried by a number of antigens, in which case the specific binding member carrying the antigen-binding site will be able to bind to the various antigens carrying the epitope.

Preferred antibodies for detection of clusterin include anti-ApoJ mouse polyclonal antibody from Abcam pn AB349-50 and the clusterin human, mouse monoclonal antibody, clone: Hs-3, Cat. No. RD182034110-H3 from BioVendor.

### Clusterin-Derived Peptides

As used herein with reference to all aspects of the invention, the term “clusterin-derived peptides” refers to one or more polypeptide species containing 5 or more (such as 6, 7, 8, 9, 10, 15, 20, 25, or more) amino acids that have at least 70%, 80%, 90%, 95%, 99% or 100% identity to the human clusterin sequence disclosed in UniProt Accession No. P10909, sequence version 1 and GI No. 116533 or such appropriate homologue of clusterin in non-human subjects (e.g. the murine clusterin protein sequence disclosed herein). The clusterin-derived peptides may be produced synthetically using methods well known in the art such as solid-phase peptide synthesis or may be naturally produced by enzymatic digestion of recombinant clusterin or a natural source containing clusterin such as human plasma or brain tissue. Particularly advantageous clusterin-derived peptides for measuring clusterin levels in humans are peptides TLLSNLEEAK, ASSIIDELFQDR, IDSLLENDR, VTTVASHTSDSDVPSGVTEVVVK, ALQEYR and YNELLK.

### Assessing and Prognosing AD

As used herein “assessing” AD includes the provision of information concerning the type or classification of the disease or of symptoms which are or may be experienced in connection with it. This specifically includes prognosis of the medical course of the disease, for example its duration, severity and the course and rate of progression from e.g. MCI or pre-symptomatic AD to clinical AD. This also includes prognosis of AD-associated brain pathology such as fibrillar amyloid burden, cortical and hippocampal atrophy and accumulation of neurofibrillary tangles. The assessment may be of an aggressive form of AD and/or a poor prognosis.

### Aggressive AD

An aggressive form of AD is generally associated with a poor prognosis, and specifically includes rapidly progressing AD, more severe cognitive impairment and/or more severe brain pathology. “Rapidly progressing AD” may be AD characterised by: a decline in a MMSE score of the subject at a rate of at least 2 MMSE points per year; and/or a decline in an ADAS-Cog score of the subject at a rate of at least 2 ADAS-Cog points per year. Both retrospective decline (i.e. the rate of decline prior to the point at which the sample is obtained from the subject) and prospective decline (i.e. the rate of decline following the point at which the sample is obtained from the subject) are specifically contemplated.

### Screening Methods

Test agents, such as pharmaceutically active compounds, antibodies (including antibodies and fragments thereof that selectively bind clusterin), agents that inhibit clusterin gene expression (including antisense, ribozyme, siRNA and triple helix that silence or downregulate clusterin transcription and/or translation) may be assessed for the ability to alleviate AD in a subject. The screening methods contemplated herein include in vivo assays for agents that affect the level of clusterin, e.g. as determined by measuring clusterin in a biological sample obtained from the subject. A test agent that is found to affect (in particular decrease) clusterin levels and/or which is able to slow, halt or reverse the progress of AD in the subject may be identified as therapeutically useful for treatment of AD. In particular, the test agent may be identified as being able to shift a rapidly progressing AD towards a non-rapidly progressing AD.

A test agent that is identified as inhibiting or reducing clusterin, slowing, halting or reversing the progress of AD in the subject may be formulated into a pharmaceutically acceptable composition. The pharmaceutically acceptable composition may be used in a method of treatment of a subject having AD or a prophylactic method of treating a subject at risk of developing AD. For example, antibodies and fragments thereof that selectively bind clusterin and/or agents that inhibit clusterin gene expression (including antisense, ribozyme, siRNA and triple helix that silence or down-regulate clusterin transcription and/or translation) may find use in the such methods of treatment.

The following is presented by way of example and is not to be construed as a limitation to the scope of the claims.

### EXAMPLES

As a proxy measure of in vivo pathology we used structural neuroimaging of the degree of atrophy in the hippocampus and entorhinal cortex (ERC), two distinct and key components of the medial temporal lobe (MTL) that show early pathological changes in AD [13]. For rate of clinical progression we used both retrospective and prospective measures of cognitive decline.

We first performed two independent discovery-phase studies using proteomic analysis of plasma in separate groups of subjects. In the first, we sought proteins reflecting hippocampal atrophy, as a measure of the extent of disease, in MCI and established AD. In the second, we identified proteins differentially expressed in fast progressing AD patients relative to those with a less aggressive disease course. Our aim in these discovery-phase studies was to identify plasma proteins common to both paradigms. We then validated these candidate biomarkers using specific immunoassays in a large independent cohort of AD, MCI and control subjects. For the validation studies, we selected automated measurements of the entorhinal cortex, as an alternative neuroimaging measure of disease pathology.

### Materials and Methods

### Samples and Subjects

Samples used came from two studies—the Alzheimer's Research Trust funded cohort at KCL (KCL ART) [5] and AddNeuroMed [2] studies. The KCL ART study is a cohort of people with ad, MCI and normal elderly started in 2001 for the purpose of biomarker discovery and validation. All subjects were white UK citizens with grandparents born in the UK and are assessed annually. AddNeuroMed is a cross-European cohort founded for biomarker discovery; AD cases are assessed 3 monthly in the first year and annually thereafter, MCI and control groups are assessed annually. All subjects are white Europeans recruited from UK, France, Italy, Finland, Poland and Greece. Cases with probable AD according to NINCDS-ADRDA criteria were recruited through secondary care as previously described [5] and evaluated with a standardised assessment previously shown to have high diagnostic validity against assessment at post mortem [30]. The full standardized assessment includes demographic and medical information, cognitive assessment including MMSE (both studies), ADAS-Cog (AddNeuroMed only) and CERAD battery, and scales to assess function, behaviour and global levels of severity including the CDR. Cases with amnestic MCI were defined as subjective memory complaint, CDR score <1 and evidence for objective memory impairment using the CERAD delayed word list recall (−1.5 SD cut off). MCI cases were recruited from both primary and secondary care. Normal elderly controls, defined as having no evidence of cognitive impairment, were recruited systematically from primary care patient lists in the case of the KCL ART study and from primary care and from elsewhere in the AddNeuroMed study.

### Discovery-Phase Proteomic Experiments (Hippocampal Atrophy)

For both discovery-phase studies, plasma samples from selected subjects from the AddNeuroMed or KCL-ART cohorts were analysed by 2DGE followed by tandem mass spectrometry as previously described [5].

### Subjects and Samples

We examined samples from the KCL-ART cohort from 27 patients with mild to moderate AD (target MMSE>15) and 14 subjects with amnestic MCI [31]. Cases with probable AD according to NINCDS-ADRDA criteria and amnestic MCI were identified as previously described [5] and evaluated with a standardised assessment previously shown to have high diagnostic validity [30]. This study was approved by the South London and Maudsley NHS Foundation Trust ethics committee.

### MRI Data Acquisition

Whole-brain coronal three-dimensional SPGR images (repetition time [TR]=14 msec, echo time [TE]=3 msec, 256×192 acquisition matrix, 124-mm×1.5-mm slices) were obtained on a GE Signa 1.5T Neuro-optimized MR system.

### 2DGE Analysis

Gels were analysed using Melanie 2-D software and detected spots matched using landmarked proteins to derive a synthetic image for both AD and MCI groups. The two synthetic gels were merged resulting in 296 spots matched across the whole cohort. The optical density of each spot was normalised to the total optical density of all spots on a gel. Spot data were scaled to unit-variance and log₁₀ transformed where appropriate. Observations with greater than 50% missing values were excluded. The PLS model took the scaled spot volumes as predictor variables and hippocampal volumes (left and right) as the response variables.

### Discovery-Phase Proteomic Experiments (Fast Versus Slow Progressors)

### Subjects and Samples

We examined samples from 51 subjects with mild-moderate AD (NINCDS-ADRDA criteria; target MMSE>10) from the AddNeuroMed cohort. Subjects were assessed by clinical examination at baseline and 3-monthly intervals over a 1-year period. Patients were characterised as fast progressors based on a decline of 2 or more points on the Alzheimer disease assessment scale—Cognitive (ADAS-Cog) score from baseline to the 6-month follow-up time point. Using this criterion, we characterised 22 subjects as ‘fast’ and 29 as ‘slow’ progressors. As shown in Table 1, the two groups were well matched by age, gender and baseline ADAS-Cog and MMSE scores (see mean and std. deviation values in Table 1). All subjects in both groups were on acetylcholinesterase inhibitor treatment. Plasma samples used for the proteomic experiments were obtained at the baseline evaluation.

### 2DGE Analysis

Progenesis SameSpots v3.0 (Nonlinear Dynamics) was used for image analysis. Prominent spots were used to manually assign vectors to each gel image. The vectors were used to warp the images and align the spot positions to a common reference gel. Spot detection was performed on this reference gel after editing and removing artefacts. We successfully matched 413 spots across every gel between the two subject groups. Partial least squares discriminant analysis (PLS-DA) was used to derive a panel of protein spots that discriminated between fast and slow progressor groups of AD patients.

### Mass Spectrometry

Protein spots of interest were excised, washed and in-gel digested with trypsin. Peptides were extracted by acetonitrile and aqueous washes and analysed by LC/MS/MS as previously reported [5]. The mass spectral data were processed into peptide peak lists and searched against the Swiss-Prot Database using Mascot software (Matrix Science, UK).

### Validation-Phase Experiments: Imaging Measures of Brain Atrophy

### MRI Data Acquisition

Whole-brain sagittal three-dimensional MP-RAGE images (TR=8.6, TE=3.8, 256×192 acquisition matrix, 180×1.2 mm slices) were obtained from all subjects on a 1.5T MR system at each of the 6 centres. Regular quality control was undertaken using the ADNI phantom and two volunteers who visited each of the centres, in order to ensure compatible images across the study. Entorhinal cortical thickness was calculated using a cortical reconstruction technique developed by Fischl and Dale [32, 33]. This included removal of non-brain tissue using a hybrid watershed/surface deformation procedure, automated Talairach transformation, intensity normalisation, tessellation of the gray matter white matter boundary, automated topology correction, and surface deformation following intensity gradients to optimally place the gray/white and gray/cerebrospinal fluid borders at the location where the greatest shift in intensity defines the transition to the other tissue class. Surface inflation was followed by registration to a spherical atlas which utilized individual cortical folding patterns to match cortical geometry across subjects and parcellation of the cerebral cortex into units based on gyral and sulcal structure.

### Validation-Phase Experiments: Fast Versus Slow AD Progressors

### Subjects and Samples

We used samples from AD patients from both the AddNeuroMed (N=228) and KCL-ART (N=117) studies for these experiments. Since ADAS-Cog scores were not obtained in the KCL-ART study, we used rate of decline in MMSE scores for classification of fast and slow progressors. We used MMSE scores obtained at the time of blood sampling to derive an annualised retrospective progression rate in order to stratify AD patients into fast and slow progressors by using the equation: Progression rate=(30)−(MMSE score at the time of blood sampling)/duration of illness in years. We defined fast progressors as those with a decline of 2 or more MMSE points per year.

Similarly, we also calculated an annualised prospective progression rate in the combined AddNeuroMed and KCL-ART cohorts by calculating the decline in MMSE score one year after blood sampling. We defined fast progressors as those with a decline of more than 2 MMSE points per year.

### Clusterin ELISA Assay

Plasma concentration of clusterin was assayed by a commercially available ELISA kit (Human Clusterin ELISA, RD194034200R, Biovendor Laboratory Medicine Inc). All samples were run in duplicate.

### Gene Expression of Clusterin

### RNA Extraction

Approximately 2.5 ml of venous blood was collected into a PAXgene tube for each subject at the baseline visit and stored at −20° C. overnight before being transferred to −80° C. storage. The evening prior to RNA extraction tubes were removed from storage and left at room temperature to thaw. RNA was extracted using the PAXgene Blood RNA kit according to manufacturer's instructions. Following extraction, the samples were assessed for yield using a spectrophotometer and quality using the RNA 6000 Pico Chip on the Agilent Bioanalyser. Samples with a RNA integrity number (RIN) >7.0 were used for PCR assays.

cDNA Synthesis and Real-Time PCR

Using the Quantitect Reverse Transcription kit (Qiagen), 500 ng RNA was reverse transcribed to cDNA in a 40 μl reaction, which was subsequently diluted to 200 μl. RT-PCR reactions were performed in 384 well plates in the 7900HT Fast Real-time PCR machine from Applied Biosystems. In brief, each well contained 2.5 μl Quantifast SYBR green PCR mix (Qiagen), 1.25 μl nuclease-free water, 0.25 μl Primer Mix (Specific for each gene and designed by Primer Design Ltd) and 1 μl diluted cDNA (corresponding to 2.5 ng RNA input). geNORM housekeeping selection kit (Primer Design Ltd) was used to assay a panel of 12 housekeeping genes in a subset of the samples. The data was analysed using the freely available software NormFinder, and the two most stable genes for normalisation were found to be splicing factor 3a, subunit 1 (SF3A1) and ATP synthase, H+ transporting, mitochondrial F1 complex, beta polypeptide (ATP5B). All samples were assayed in duplicate and a standard curve of known copy number (produced from cloned PCR products) was run on each plate for clusterin, SF3A1 and ATP5B. The standard curve was also used to determine the efficiency of the PCR reaction (90 to 110%). The number of copies of clusterin present in each sample was normalised by dividing clusterin levels by the average copy number of the two housekeeping genes and multiplying by 1000. Data were non-parametric, and were therefore log-transformed to give a normal distribution and allow analysis of co-variance and comparisons with the plasma protein levels.

### TASTPM Transgenic Mouse Model Experiments

Heterozygote transgenic mice overexpressing both hAPP695swe (TAS10) and presenilin-1 M146V mutations (TPM) were generated by standard techniques as previously described [15]. Wild type animals were of the C57/B16 line. Western blot analysis of plasma clusterin concentration was done by collecting plasma samples at 6 months of age. 10 microgrammes total protein was loaded in sample buffer. Primary antibody was anti-ApoJ mouse polyclonal from Abcam pn AB349-50 used at 1:5,000 dilution. Immunohistochemical studies to quantify brain amyloid burden were performed as previously described [15]. The animal experiments were conducted according to the Council of Europe (Directive 86/609) and Danish guidelines.

### Example 1

### Proteomic Identification of Plasma Proteins Associated with Hippocampal Atrophy and Rapid Clinical Progression in AD

Using 2DGE and LC-MS/MS we identified a set of proteins in plasma that showed significant correlation with hippocampal atrophy in a combined group of 44 subjects representing a continuum of disease; 27 with mild to moderate AD and 17 with MCI (see Table 1 below).

TABLE 1
Discovery-phase Subjects

| | | | | | Rate of clinical | | | |
|---|---|---|---|---|---|---|---|---|
| | Hippocampal volume study | | | | progression study | | | |
| | | | | | Rapid | | Non-rapid | |
| | AD | | MCI | | decliners | | decliners | |
| | (n = 27) | | (n = 17) | | (n = 22) | | (n = 29) | |
| Gender (M/F) | 9/18 | | 7/10 | | 9/13 | | 11/18 | |
| Age (years) | 78 | (4.0) | 77 | (6.0) | 76 | (7.1) | 79 | (6.8) |
| Disease | 4.8 | (3.5) | n/a | | 4.1 | (3.3) | 5.0 | (4.0) |
| duration (years) | | | | | | | | |
| MMSE | 22 | (3.9) ^{‡} | 26 | (2.1) | 20.7 | (4.3) | 20.9 | (5.2) |

| | | | | | | |
|---|---|---|---|---|---|---|
| Total | 5.82 | (1.40) * | 6.96 | (1.25) | n/a | n/a |
| hippocampal | | | | | | |
| volume (mL) | | | | | | |

| | | | | | | |
|---|---|---|---|---|---|---|
| Rate of | n/a | n/a | 7.95 | (5.2) ^{§} | −3.3 | (4.5) |
| decline in | | | | | | |
| ADAS-Cog score | | | | | | |

Values are expressed as mean ± (SD)
^{‡} differs from MCI; p < 0.001
* differs from MCI; p = 0.01
^{§} Differs from non-rapid decliners; p < 0.001

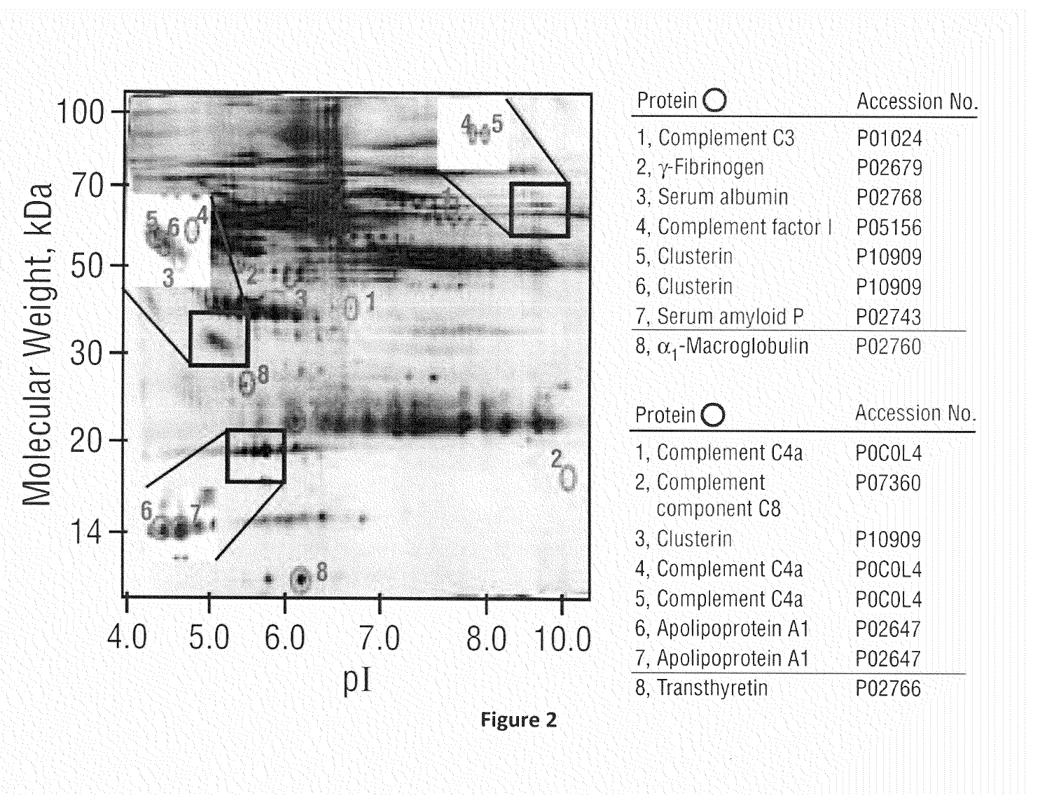

Bivariate correlation of integrated optical densities of spots detected by 2DGE revealed 13 that were significantly associated with hippocampal volume (r≧+/−0.35 and p<0.05) in the combined cohort of AD and MCI subjects. Subsequently, using partial least squares (PLS) regression [14], a method particularly suited to analysis of proteomic data where collinearity of predictor variables is common, a model with two components was fitted to the hippocampal volume data. This model was constituted by 8 of the 13 spots which, together, explained 34% of the variance (R2Y) in hippocampal volume. Using LC-MS/MS we determined that these eight 2DGE spots represented complement C3, γ-fibrinogen, serum albumin, complement factor-I (CFI), clusterin (present in two spots), α-1-microglobulin, and serum amyloid-P (SAP) (FIG. 2). We then performed a second 2DGE/mass spectrometry experiment in an independent set of samples—this time in 51 carefully matched (age, gender, severity at the time of blood sampling) AD patients that we could divide into fast or slow progressors based on their annualized rate of cognitive decline (see Table 1). A PLS model that could discriminate the fast from slow progressing AD groups was constituted by the integrated optical densities of 27 silver-stained 2DGE spots. Of these, 8 were well-defined, discrete and present in all 51 gels and were identified by LC-MS/MS. These spots contained complement component C4 (present in three spots), complement C8, clusterin, apolipoprotein-A1 (present in two spots) and transthyretin (FIG. 2).

### Example 2

### Clusterin is Associated with Atrophy of the Entorhinal Cortex, Severity of Cognitive Impairment and Speed of Progression in AD

Only one protein was identified as a potential AD biomarker from both discovery phase studies—clusterin. We therefore validated the plasma clusterin finding in a large cohort of 689 subjects; 344 with neuroimaging from the AddNeuroMed study (119 with AD, 115 with MCI and 110 controls) and 345 with AD from a long term biomarker study—the KCL ART cohort (see Tables 2a and 2b below).

TABLE 2a
Validation-phase Subjects: AddNeuroMed
cohort (Neuro-imaging studies)

| | AD | | MCI | | Control | |
|---|---|---|---|---|---|---|
| | (n = 119) | | (n = 115) | | (n = 110) | |
| Gender (M/F) | 41/78 | | 59/56 | | 50/60 | |
| Age (years) | 75.6 | (6.4) * | 74.5 | (5.6) | 72.9 | (6.7) |
| MMSE | 20.8 | (4.7) ^{§ ‡} | 26.9 | (3.0) ^{§} | 29.1 | (1.2) |
| ERC thick- | 2.6 | (0.52) ^{§, ‡} | 3.0 | (0.49) ^{§} | 3.3 | (0.35) |
| ness (mm) | | | | | | |

Values are expressed as mean ± (SD)
* Differs from control; p = 0.003
^{§} Differs from control; p < 0.001
^{‡} Differs from MCI; p < 0.001

TABLE 2b
Combined ART and AddNeuromed Cohorts - AD fast vs. AD slow decliners

| | Retrospective decline | | | | Prospective decline | | | |
|---|---|---|---|---|---|---|---|---|
| | Fast | | Slow | | Fast | | Slow | |
| | decliners | | decliners | | decliners | | decliners | |
| | (n = 219) | | (n = 125) | | (n = 115) | | (n = 122) | |
| Sex (M/F) | 74/145 | | 54/71 | | 43/72 | | 47/75 | |
| Age (years) | 78.0 | (6.2) | 77.7 | (6.4) | 77.7 | (6.3) | 77.5 | (6.4) |
| Rate of | 4.5 | (2.7) | 1.1 | (1.0) * | 5.0 | (3.2) | −0.9 | (2.0) * |
| decline in | | | | | | | | |
| MMSE per year | | | | | | | | |
| Disease | 3.9 | (2.4) | 6.4 | (3.8) * | 4.7 | (3.3) | 4.0 | (3.3) |
| duration (years) | | | | | | | | |

Values are expressed as mean ± (SD)
* p < 0.001

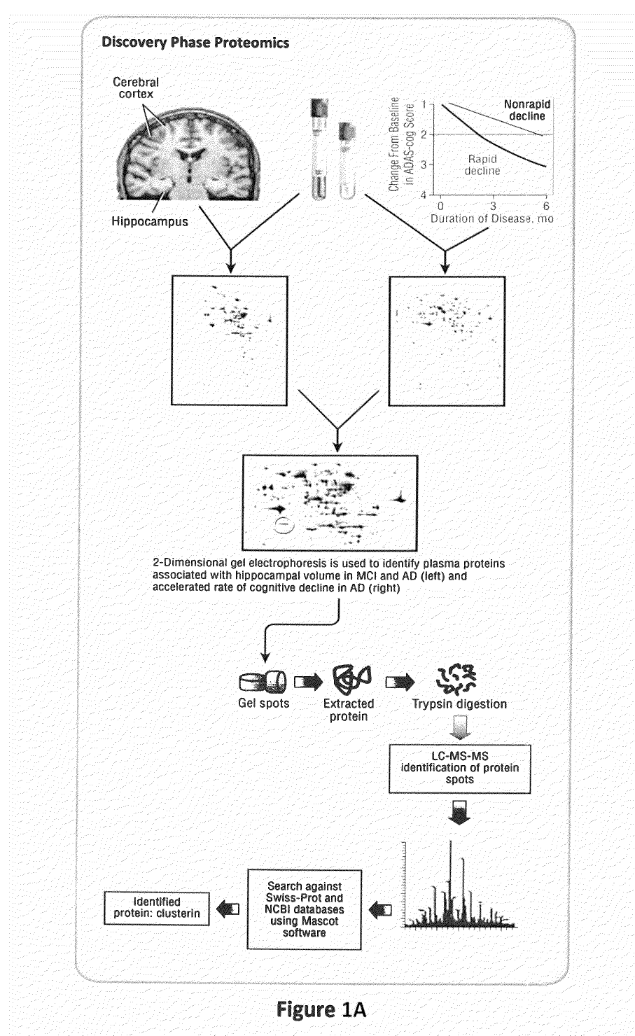

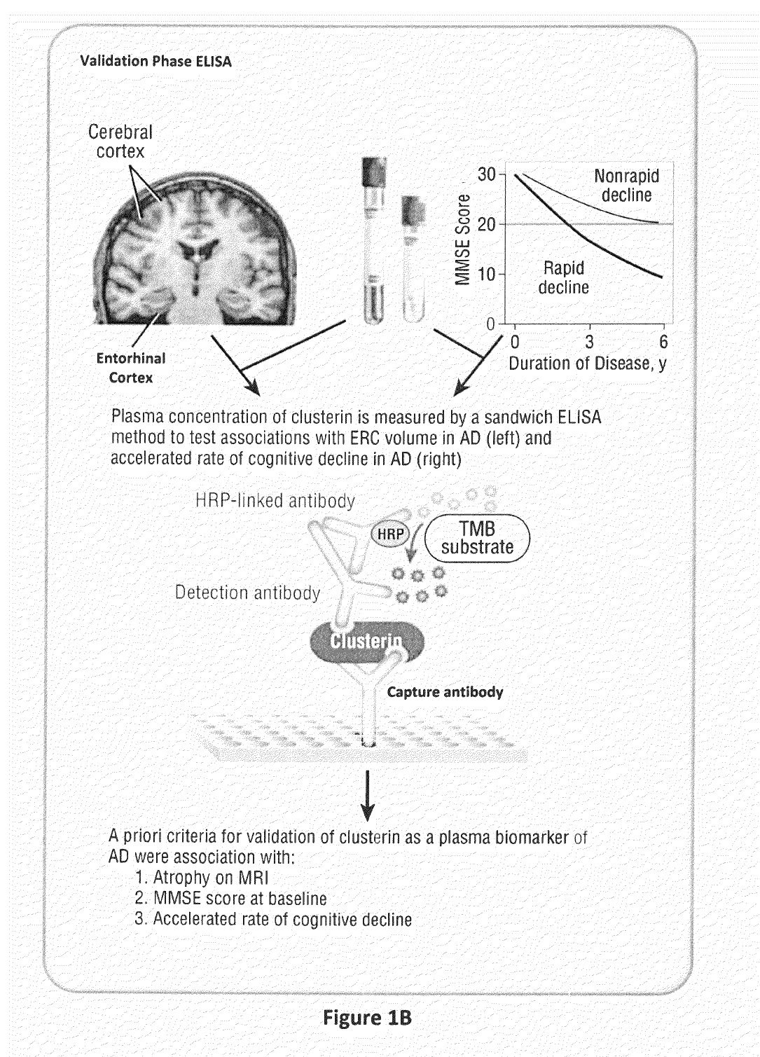

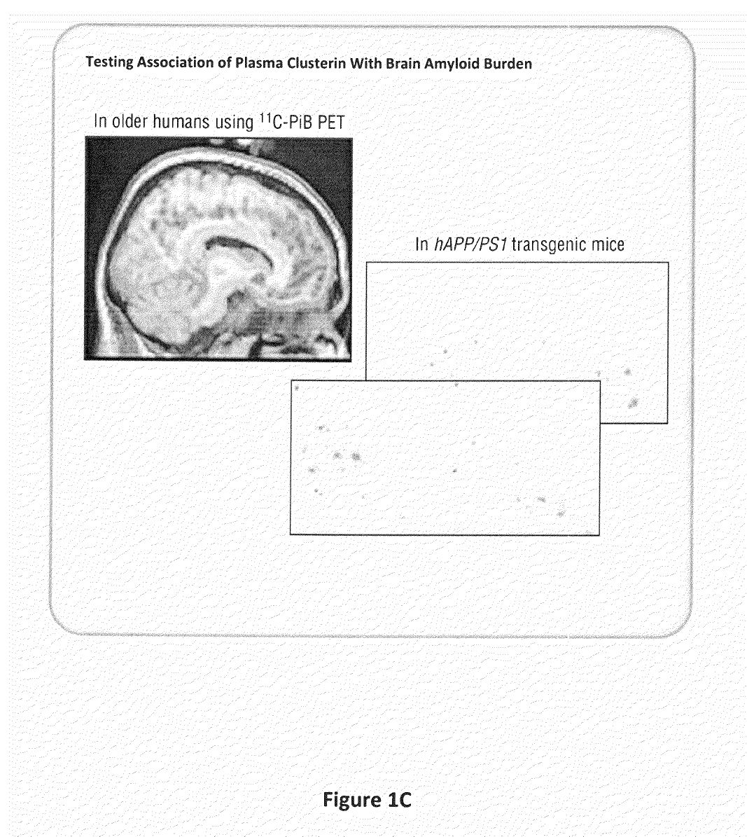

We used atrophy in the entorhinal cortex (ERC) as a neuroimaging measure of disease pathology (see FIG. 1).

We defined a priori, criteria for validation of clusterin as an AD biomarker as:
1. Significant association between plasma concentration and evidence of atrophy on MRI;
2. Significant association between plasma concentration and MMSE score in patients with AD; and
3. Increase in plasma concentration in fast progressing AD patients relative to slow progressing and hence less aggressive disease.

Confirming the discovery-phase study, we observed a significant association between clusterin concentration and ERC atrophy in the combined AD+MCI cohort (n=220, R=−0.14 and p=0.04) after covarying for age. This relationship seemed to be driven primarily by association between ERC atrophy and clusterin concentration in AD patients (n=113, R=−0.31 and p=0.001). We also correlated plasma clusterin with the MMSE score—a measure of cognition available in 576 subjects with MCI and AD—and again found a highly significant negative correlation (r=−0.22; p<0.001; age as covariate).

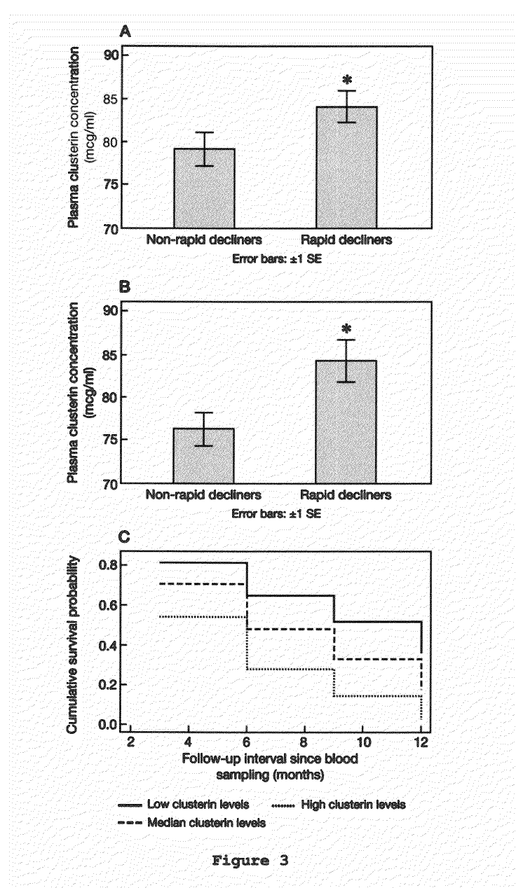

We then compared clusterin levels in fast declining AD patients relative to slow decliners using both retrospective and prospective measures of decline relative to the time of blood sampling (see FIG. 1 and Table 2b). Retrospective decline was estimated from the duration of disease and the MMSE at the point of blood sampling allowing the annualized fall in MMSE to be calculated. Prospective decline was directly measured as the fall in MMSE one year after blood sampling. We observed a significant increase in clusterin concentration in AD patients with accelerated cognitive decline prior to blood sampling (ANCOVA; n=344; t(341)=3.40; p=0.0007; duration of disease as covariate) (FIG. 3A) and an increase in clusterin concentration in AD patients with faster cognitive decline subsequent to blood sampling (N=237; independent samples t-test, p=0.01) (FIG. 3B). A Cox proportional regression analysis showed that higher plasma clusterin concentration was also associated with a greater risk of rapid cognitive decline one year after blood sampling (FIG. 3C).

### Example 3

### Gene Expression of Clusterin is Altered in AD

In order to investigate the possible mechanisms underlying the observed associations between plasma concentration of clusterin and both imaging measures of atrophy and accelerated clinical progression, we measured clusterin mRNA levels in blood cells from AD patients, MCI subjects and controls (see Table 3 below).

TABLE 3
Characteristics of subjects included in
the clusterin gene expression study

| | AD | | MCI | Control |
|---|---|---|---|---|
| | (n = 182) | | (n = 179) | (n = 207) |
| Gender (M/F) | 59/123 | | 79/100 | 83/123 |
| Age (years) | 77.2 | (6.8)*^{‡} | 75.3 (6.2) | 73.7 (7.1) |
| Disease | 4.4 | (3.0) | | |
| duration (years) | | | | |
| MMSE | 20.6 | (4.9)^{‡§} | 27.0 (2.8)^{§} | 28.5 (3.2) |

Values are expressed as mean ± (SD)
*Differs from control; p = 0.005
^{‡}Differs from MCI; p < 0.001
^{§}Differs from control; p < 0.001

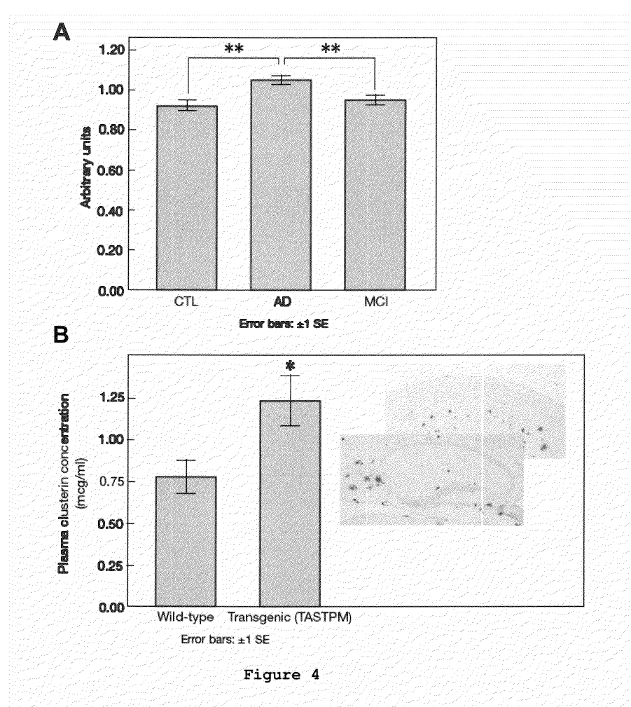

Diagnosis had a significant effect on clusterin gene expression (ANCOVA; df=2; P<0.001 and age as covariate). Pairwise comparisons between the three groups showed significantly higher clusterin gene expression in AD than MCI and control subjects (P=0.008 and P<0.001 respectively; Bonferroni adjustment for multiple comparisons) (FIG. 4A). Gender and the presence of the apolipoprotein-E (APOE) ε4 allele did not have a significant effect on clusterin mRNA levels. We did not observe a significant association between clusterin mRNA in blood cells and plasma concentration of clusterin protein.

### Example 4

### Plasma Concentration of Clusterin is Increased in hAPP695swe and Presenilin-1 M146V Overexpressing Transgenic Mice

In order to further confirm the role of clusterin as a biologically relevant peripheral biomarker of AD associated with amyloid deposition in the brain, we examined the plasma concentration of clusterin in a transgenic mouse model of AD. Double mutant TASTPM mice overexpress the hAPP695swe and presenilin-1 M146V mutations resulting in over-production of human APP and beta amyloid protein [15]. These mice mimic various hallmarks of AD such as high levels of circulating Aβ and its deposition in the form of plaques as well as cognitive and behavioural deficits [15, 16]. In the light of our MRI data in AD patients indicating associations between plasma concentration of clusterin and AD-related neuropathology, we hypothesized that plasma clusterin concentration in transgenic TASTPM mice would be higher than wild type controls. As predicted, we observed a significantly greater plasma concentration of clusterin (p=0.02; independent samples t-test) in 6-month old transgenic TASTPM mice (N=10) relative to wild-type littermates (N=10) (FIG. 4B). Previous studies have established both marked Aβ cerebral deposits as well as cognitive deficits in TASTPM mice at this age in comparison to wild type littermates [15, 16].

### Discussion

As will be appreciated from the preceding description, the present invention combines a proteomic and neuroimaging approach in a novel biomarker discovery paradigm to identify clusterin as a plasma biomarker of in vivo pathology, disease severity and clinical progression in patients with Alzheimer's disease. Most biomarker discovery studies use the presence or absence of disease as the primary outcome variable. However this binary distinction (disease/no disease) may result in the discovery of biomarkers that show excellent diagnostic characteristics but lack sensitivity in relation to disease progression or severity. In order to overcome these limitations of standard biomarker discovery studies, we sought to discover using proteomics, biomarkers for aggressive AD where the primary outcomes were association with both atrophy of the medial temporal lobe (MTL), a well-established neuroimaging measure of disease pathology, and the rate of progression of cognitive decline. In the discovery phase, we used hippocampal atrophy derived from manual tracing of the hippocampal formation from MRI images and in the much larger validation phase, automated regional analysis of the entorhinal cortex, an adjacent region of the medial temporal lobe and the site of earliest pathology in AD. For the discovery phase studies we combined both MCI and AD cases with the underlying assumption that amnestic MCI represents in most cases an early prodromal state. By representing a range of hippocampal volumes from incipient disease (MCI) to established AD, we reasoned that the combined AD and MCI cohort represented the most powerful subject group to identify candidate biomarkers reflecting the extent of MTL pathology.

Hippocampal atrophy is an early event in the pathogenesis of AD, is associated with an increased risk of conversion from MCI to AD and may even precede the development of cognitive decline [17, 18]. CSF levels of phospho-tau, an established biomarker for AD, correlate with hippocampal volume, indicating that this biomarker reflects an integral feature of AD pathology [19] Moreover, decreased hippocampal volume in AD patients has been shown to reflect neuronal loss, thereby confirming the validity of this measure as a marker of neurodegeneration [20]. A second independent outcome measure in the discovery-phase studies was rate of cognitive decline, which was derived as a measure of decrease in the ADAS-cog scores over a 6-month interval in patients with AD. Using this measure, we dichotomised AD patients as fast and slow decliners; an approach previously shown to predict long term prognosis in AD [21].

Our selection of a candidate biomarker for further validation was based upon association with both imaging measures of medial temporal lobe atrophy as well as with clinical progression in the discovery-phase proteomic experiments. Only clusterin was associated both with hippocampal atrophy, or extent of disease, in AD and MCI subjects and with fast progressing, or more aggressive AD. Several lines of evidence including those from human CSF, post-mortem brain and transgenic animal models suggest a plausible link between clusterin and AD pathology [22-25]. We therefore selected clusterin as a candidate plasma biomarker of AD for further validation.

We confirmed all three a priori criteria for validation of clusterin as an AD biomarker, finding robust associations with atrophy of the entorhinal cortex, with MMSE and with rate of progression (p=0.001, p<0.001 and p=0.0007 respectively). Finally, we demonstrated a significantly higher plasma concentration of clusterin in transgenic TASTPM mice overexpressing APP/PS1 mutations. Taken together, these results suggest that plasma clusterin is a biologically relevant peripheral biomarker of in vivo pathology, disease severity and clinical progression in AD. The observation that clusterin mRNA is significantly increased in blood cells in AD indicates the capacity for the clusterin gene to respond to AD associated pathology in peripheral tissue by increasing expression. It is interesting to note that several previous studies suggest that the clusterin gene is a highly sensitive biosensor to reactive oxygen species implicated in aging and age-related diseases [26]. Our findings demonstrating altered regulation of clusterin gene expression in AD therefore suggest a primary role for clusterin in the disease process. While the increase in clusterin mRNA in AD patients does not correlate directly with plasma clusterin concentration, it suggests that the primary source of plasma clusterin that we find predictive of more aggressive disease is derived predominantly from organs other than blood cells such as the liver, or possibly even the brain.

Although our present results do not directly address the mechanisms underlying the role of clusterin in AD pathogenesis, previous studies have suggested that it belongs to a family of extracellular chaperone proteins regulating amyloid formation and clearance [27]. While its precise role in regulating in vivo amyloidogenesis remains unclear, in vitro studies show that clusterin regulates amyloid formation in a biphasic manner with low clusterin:substrate ratios enhancing and higher ratios inhibiting amyloid formation respectively [28]. In mice, in vivo binding of Aβ to clusterin enhances its clearance and efflux through the blood brain barrier [29]. However, previous reports on clusterin as a candidate AD biomarker have been inconclusive [23, 25].

In summary, we have employed a novel proteomic-neuroimaging discovery paradigm where the primary endpoints were well-established measures of pathology in the medial temporal lobe and rate of disease progression, rather than reliance on distinction of AD patients from controls. We first identified clusterin as a candidate plasma biomarker and subsequently validated this finding in a large independent cohort of AD patients using quantitative immunoassays. We believe that these data hold considerable promise for clinical utility of clusterin as a biologically relevant peripheral biomarker of Alzheimer's disease.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. Cummings, J. L., Doody, R., and Clark, C. 2007. Disease-modifying therapies for Alzheimer disease: challenges to early intervention. Neurology 69:1622-1634.
2. Lovestone, S., Francis, P., and Strandgaard, K. 2007. Biomarkers for disease modification trials—the innovative medicines initiative and AddNeuroMed. J Nutr Health Aging 11:359-361.
3. Mueller, S. G., Weiner, M. W., Thal, L. J., Petersen, R. C., Jack, C. R., Jagust, W., Trojanowski, J. Q., Toga, A. W., and Beckett, L. 2005. Ways toward an early diagnosis in Alzheimer's disease: The Alzheimer's Disease Neuroimaging Initiative (ADNI). Alzheimers Dement 1:55-66.
4. Hampel, H., Burger, K., Teipel, S. J., Bokde, A. L., Zetterberg, H., and Blennow, K. 2008. Core candidate neurochemical and imaging biomarkers of Alzheimer's disease. Alzheimers Dement 4:38-48.
5. Hye, A., Lynham, S., Thambisetty, M., Causevic, M., Campbell, J., Byers, H. L., Hooper, C., Rijsdijk, F., Tabrizi, S. J., Banner, S., et al. 2006. Proteome-based plasma biomarkers for Alzheimer's disease. Brain 129:3042-3050.
6. Thambisetty, M., Hye, A., Foy, C., Daly, E., Glover, A., Cooper, A., Simmons, A., Murphy, D., and Lovestone, S. 2008. Proteome-based identification of plasma proteins associated with hippocampal metabolism in early Alzheimer's disease. J Neurol 255:1712-1720.
7. Akuffo, E. L., Davis, J. B., Fox, S. M., Gloger, I. S., Hosford, D., Kinsey, E. E., Jones, N. A., Nock, C. M., Roses, A. D., Saunders, A. M., et al. 2008. The discovery and early validation of novel plasma biomarkers in mild-to-moderate Alzheimer's disease patients responding to treatment with rosiglitazone. Biomarkers 13:618-636.
8. Cutler, P., Akuffo, E. L., Bodnar, W. M., Briggs, D. M., Davis, J. B., Debouck, C. M., Fox, S. M., Gibson, R. A., Gormley, D. A., Holbrook, J. D., et al. 2008. Proteomic identification and early validation of complement 1 inhibitor and pigment epithelium-derived factor: Two novel biomarkers of Alzheimer's disease in human plasma. Proteomics Clinical Applications 2:467-477.
9. Ray, S., Britschgi, M., Herbert, C., Takeda-Uchimura, Y., Boxer, A., Blennow, K., Friedman, L. F., Galasko, D. R., Jutel, M., Karydas, A., et al. 2007. Classification and prediction of clinical Alzheimer's diagnosis based on plasma signaling proteins. Nat Med 13:1359-1362.
10. 1998. Consensus report of the Working Group on: “Molecular and Biochemical Markers of Alzheimer's Disease”. The Ronald and Nancy Reagan Research Institute of the Alzheimer's Association and the National Institute on Aging Working Group. Neurobiol Aging 19:109-116.
11. Brooks, J. O., 3rd, and Yesavage, J. A. 1995. Identification of fast and slow decliners in Alzheimer disease: a different approach. Alzheimer Dis Assoc Disord 9 Suppl 1:519-25.
12. Kraemer, H. C., Tinklenberg, J., and Yesavage, J. A. 1994. ‘How far’ vs ‘how fast’ in Alzheimer's disease. The question revisited. Arch Neurol 51:275-279.
13. Braak, H., and Braak, E. 1991. Neuropathological stageing of Alzheimer-related changes. Acta Neuropathol 82:239-259.
14. Eriksson, L., Antti, H., Gottfries, J., Holmes, E., Johansson, E., Lindgren, F., Long, I., Lundstedt, T., Trygg, J., and Wold, S. 2004. Using chemometrics for navigating in the large data sets of genomics, proteomics, and metabonomics (gpm). Anal Bioanal Chem 380:419-429.
15. Howlett, D. R., Richardson, J. C., Austin, A., Parsons, A. A., Bate, S. T., Davies, D. C., and Gonzalez, M. I. 2004. Cognitive correlates of Abeta deposition in male and female mice bearing amyloid precursor protein and presenilin-1 mutant transgenes. Brain Res 1017:130-136.
16. Howlett, D. R., Bowler, K., Soden, P. E., Riddell, D., Davis, J. B., Richardson, J. C., Burbidge, S. A., Gonzalez, M. I., Irving, E. A., Lawman, A., et al. 2008. Abeta deposition and related pathology in an APP×PS1 transgenic mouse model of Alzheimer's disease. Histol Histopathol 23:67-76.
17. Kantarci, K., and Jack, C. R., Jr. 2003. Neuroimaging in Alzheimer disease: an evidence-based review. Neuroimaging Clin N Am 13:197-209.
18. Nestor, P. J., Scheltens, P., and Hodges, J. R. 2004. Advances in the early detection of Alzheimer's disease. Nat Med 10 Suppl:S34-41.
19. Hampel, H., Burger, K., Pruessner, J. C., Zinkowski, R., DeBernardis, J., Kerkman, D., Leinsinger, G., Evans, A. C., Davies, P., Moller, H. J., et al. 2005. Correlation of cerebrospinal fluid levels of tau protein phosphorylated at threonine 231 with rates of hippocampal atrophy in Alzheimer disease. Arch Neurol 62:770-773.
20. Bobinski, M., de Leon, M. J., Wegiel, J., Desanti, S., Convit, A., Saint Louis, L. A., Rusinek, H., and Wisniewski, H. M. 2000. The histological validation of post mortem magnetic resonance imaging-determined hippocampal volume in Alzheimer's disease. Neuroscience 95:721-725.
21. Helmer, C., Andrieu, S., Peres, K., Orgogozo, J. M., Vellas, B., and Dartigues, J. F. 2007. Predictive value of 6-month decline in ADAS-cog for survival without severe Alzheimer's disease. Dement Geriatr Cogn Disord 23:168-174.
22. DeMattos, R. B., O'Dell M, A., Parsadanian, M., Taylor, J. W., Harmony, J. A., Bales, K. R., Paul, S. M., Aronow, B. J., and Holtzman, D. M. 2002. Clusterin promotes amyloid plaque formation and is critical for neuritic toxicity in a mouse model of Alzheimer's disease. Proc Natl Acad Sci USA 99:10843-10848.
23. Lidstrom, A. M., Bogdanovic, N., Hesse, C., Volkman, I., Davidsson, P., and Blennow, K. 1998. Clusterin (apolipoprotein J) protein levels are increased in hippocampus and in frontal cortex in Alzheimer's disease. Exp Neurol 154:511-521.
24. Nilselid, A. M., Davidsson, P., Nagga, K., Andreasen, N., Fredman, P., and Blennow, K. 2006. Clusterin in cerebrospinal fluid: analysis of carbohydrates and quantification of native and glycosylated forms. Neurochem Int 48:718-728.
25. Sihlbom, C., Davidsson, P., Sjogren, M., Wahlund, L. O., and Nilsson, C. L. 2008. Structural and quantitative comparison of cerebrospinal fluid glycoproteins in Alzheimer's disease patients and healthy individuals. Neurochem Res 33:1332-1340.
26. Trougakos, I. P., and Gonos, E. S. 2006. Regulation of clusterin/apolipoprotein J, a functional homologue to the small heat shock proteins, by oxidative stress in ageing and age-related diseases. Free Radic Res 40:1324-1334.
27. Wilson, M. R., Yerbury, J. J., and Poon, S. 2008. Potential roles of abundant extracellular chaperones in the control of amyloid formation and toxicity. Mol Biosyst 4:42-52.
28. Yerbury, J. J., Poon, S., Meehan, S., Thompson, B., Kumita, J. R., Dobson, C. M., and Wilson, M. R. 2007. The extracellular chaperone clusterin influences amyloid formation and toxicity by interacting with prefibrillar structures. Faseb J 21:2312-2322.
29. Bell, R. D., Sagare, A. P., Friedman, A. E., Bedi, G. S., Holtzman, D. M., Deane, R., and Zlokovic, B. V. 2007. Transport pathways for clearance of human Alzheimer's amyloid beta-peptide and apolipoproteins E and J in the mouse central nervous system. J Cereb Blood Flow Metab 27:909-918.
30. Foy, C. M., Nicholas, H., Hollingworth, P., Boothby, H., Willams, J., Brown, R. G., Al-Sarraj, S., and Lovestone, S. 2007. Diagnosing Alzheimer's disease—non-clinicians and computerised algorithms together are as accurate as the best clinical practice. Int J Geriatr Psychiatry 22:1154-1163.
31. Petersen, R. C., Smith, G. E., Waring, S. C., Ivnik, R. J., Tangalos, E. G., and Kokmen, E. 1999. Mild cognitive impairment: clinical characterization and outcome. Arch Neurol 56:303-308.
32. Desikan, R. S., Segonne, F., Fischl, B., Quinn, B. T., Dickerson, B. C., Blacker, D., Buckner, R. L., Dale, A. M., Maguire, R. P., Hyman, B. T., et al. 2006. An automated labeling system for subdividing the human cerebral cortex on MRI scans into gyral based regions of interest. Neuroimage 31:968-980.
33. Fischl, B., van der Kouwe, A., Destrieux, C., Halgren, E., Segonne, F., Salat, D. H., Busa, E., Seidman, L. J., Goldstein, J., Kennedy, D., et al. 2004. Automatically parcellating the human cerebral cortex. Cereb Cortex 14:11-22.
34. Harold, D., et al., 2009. Genome-wide association study identifies variants at CLU and PICALM associated with Alzheimer's disease. Nature Genetics, Advance publication online: 6 Sep. 2009; doi: 10.1038/ng.440.
35. Lambert, J-C., et al., 2009. Genome-wide association study identifies variants at CLU and CR1 associated with Alzheimer's disease. Nature Genetics, Advance publication online: 6 Sep. 2009; doi: 10.1038/ng.439.
36. Assessment Scales in Old Age Psychiatry by Alistair Burns, Brian Lawlor and Sarah Craig. Pub: Martin Dunitz, London 1999.
37. Vellas B, Andrieu S, Sampaio C, Coley N, Wilcock G. Endpoints for trials in Alzheimer's disease: a European task force consensus. Lancet Neurol 2008; 7(5):436-450.
38. Vellas B, Andrieu S, Sampaio C, Wilcock G. Disease-modifying trials in Alzheimer's disease: a European task force consensus. Lancet Neurol 2007; 6(1):56-62.

1. A prognostic method for assessing Alzheimer's disease (AD) in a subject, comprising determining an amount of clusterin in a biological sample obtained from said subject, wherein a greater amount of clusterin, compared with a pre-determined reference level, is indicative of said subject having rapidly progressing AD, more severe cognitive impairment and/or more severe brain pathology.

2. The method according to claim 1, wherein said biological sample comprises at least one of blood plasma and blood cells.

3. The method according to claim 2, wherein determining the amount of clusterin comprises quantifying the blood plasma concentration of clusterin.

4. The method according to claim 1, wherein determining the amount of clusterin comprises:
contacting said sample with at least one specific binding member that selectively binds to clusterin; and
at least one of detecting and quantifying a complex formed by said specific binding member and clusterin.

5. The method according to claim 1, wherein determining the amount of clusterin comprises measuring the level of a clusterin-encoding mRNA derived from said biological sample.

6. The method according to claim 1, wherein determining the amount of clusterin comprises measuring at least one of the level of clusterin and a clusterin-derived peptide, or a multiplicity of said peptides, by mass spectrometry.

7. The method according to claim 1, wherein said subject has previously been diagnosed with (i) AD or (ii) mild cognitive impairment.

8. The method according to claim 1, wherein said subject is a human of at least 60 years of age, optionally at least 70 or at least 80 years of age.

9. The method according to claim 1, wherein the method is for assessing AD in a plurality of subjects, and wherein the method comprises determining an amount of clusterin in a biological sample obtained from each of said plurality of subjects.

10. The method according to claim 9, wherein the plurality of subjects are stratified according to aggressiveness of AD based on the determination of said clusterin amount.

11. The method according to claim 10, wherein the plurality of subjects are divided into slow progressing AD category and fast progressing AD category based on the determination of said clusterin amount.

12. The method according to claim 1, wherein said determination of clusterin amount is used (i) to establish the treatment strategy of said subject or subjects or (ii) to monitor the success of a treatment strategy or both (i) and (ii).

13. The method according to claim 1, wherein said determination of clusterin amount indicates that said subject or subjects have at least one of rapidly progressing AD, more severe cognitive impairment and more severe brain pathology.

14. The method according to claim 1, wherein said rapidly progressing AD is characterised by: (i) a decline in a mini-mental state examination (MMSE) score of said subject at a rate of at least 2 MMSE points per year; or (ii) a decline in an AD assessment scale-cognitive (ADAS-Cog) score of said subject at a rate of at least 2 ADAS-Cog points per year or both (i) and (ii).

15. The method according to claim 1, wherein said brain pathology is selected from: fibrillar amyloid burden in the entorhinal cortex, atrophy of the entorhinal cortex and atrophy of the hippocampus.

16. The method according to claim 1, wherein said clusterin comprises an amino acid sequence having at least 90% identity to the human clusterin sequence disclosed in UniProt Accession No. P10909, sequence version 1 and GI No. 116533.

17. A method for screening a test agent to determine its usefulness in treating Alzheimer's disease (AD), the method comprising:
determining an amount of clusterin in a biological sample obtained from a test subject having at least one AD-related clinical or pathological feature, which test subject has been treated with the test agent; and
comparing the determination of said clusterin amount with a control amount, which corresponds to the amount of clusterin in a biological sample obtained from a control subject having at least one Alzheimer's disease-related clinical or pathological feature, which control subject has not been treated with the test agent,
whereby the test agent is selected or rejected according to the extent to which the test agent alters said clusterin amount relative to said control amount.

18. The method according to claim 17, wherein the test agent is found to decrease the amount of clusterin relative to said control amount.

19. The method according to claim 17, wherein the biological sample comprises blood plasma, and wherein determining the amount of clusterin comprises quantifying the blood plasma concentration of clusterin.

20. The method according to claim 17, wherein the test subject and the control subject are the same subject, and wherein said control amount corresponds to the amount of clusterin in a biological sample obtained from said subject prior to said subject being treated with the test agent.

21. The method according to claim 17, wherein the test subject has been assessed by the method according to claim 17, and wherein the assessment of the test subject indicates that the test subject has at least one of rapidly progressing AD, more severe cognitive impairment and more severe brain pathology.

22. The method according to claim 17, wherein the test subject and the control subject are human subjects with AD.

23. The method according to claim 17, wherein the test subject and the control subject are selected from: mutant amyloid precursor protein (APP) transgenic mice; presenilin-1 (PS-1) transgenic mice; and double transgenic APP/PS-1 transgenic mice.

24. The method according to claim 23, wherein the test subject and the control subject are TASTPM transgenic mice that overexpress hAPP695swe and presenilin-1 M146V.

25. A method of making a pharmaceutical composition, comprising, having identified a test agent using a method according to claim 17, the further step of manufacturing the test agent and formulating it with a pharmaceutically acceptable carrier to provide the pharmaceutical composition.

26. A kit for assessing Alzheimer's disease (AD) in a subject by a method according to claim 1, the kit comprising:
(ia) a specific binding member that selectively binds clusterin; (ib) at least one primer or probe directed to a nucleic acid sequence that encodes clusterin or which is complementary thereto; (ic) at least one standard curve comprising two or more concentrations of a clusterin-derived peptide labelled with a set of isobaric mass tags and an additional member of the same isobaric mass tag set for labelling of a subject-derived sample or a combination of two or more of (ia), (ib) and (ic);
(ii) instructions for performing a method according to claim 1; and
optionally, (iii) one or more reagents or controls for use in determining an amount of clusterin in a biological sample.

27. A kit for screening a test agent by a method according to claim 17, the kit comprising:
(ia) a specific binding member that selectively binds clusterin; (ib) at least one primer or probe directed to a nucleic acid sequence that encodes clusterin or which is complementary thereto; (ic) at least one standard curve comprising two or more concentrations of a clusterin-derived peptide labelled with a set of isobaric mass tags and an additional member of the same isobaric mass tag set for labelling of a subject-derived sample or a combination of two or more of (ia), (ib) and (ic);
(ii) instructions for performing a method according to claim 18; and optionally;
(iii) one or more reagents or controls for use in determining an amount of clusterin in a biological sample.
